(19) United States
(12) Patent Application Publication
(10) Pub. No.: US 2017/0197016 A1
(43) Pub. Date: Jul. 13, 2017

(54) Method for enhancing postoperative healing using low pressure suction apparatus

(71) Applicant: HLD Healthy Life Devices Oy, Espoo (FI)

(72) Inventors: Kalle Palomäki, Helsinki (FI); Steven Basta, Menlo Park, CA (US)

(73) Assignee: HLD Healthy Life Devices Oy, Espoo (FI)

(21) Appl. No.: 15/401,300

(22) Filed: Jan. 9, 2017

Related U.S. Application Data
(60) Provisional application No. 62/275,813, filed on Jan. 7, 2016.

(51) Int. Cl. A61M 1/00

(52) U.S. Cl. CPC A61M 1/0037; A61M 1/008; A61M 1/0066

(57) ABSTRACT

A method for enhancing postoperative healing using a low pressure suction apparatus comprising obtaining a preferably both electrically powered and portable treatment apparatus capable of contacting, via a treatment head, a target area of a patient and introducing low-pressure suction, i.e. negative pressure flow, thereto, configuring the treatment apparatus so as to introduce predefined, advantageously, pulsating pressure variation to a suction opening arranged at the treatment head, and applying the suction opening of the treatment head on the target area before the surgery, i.e. during the preoperative period, and optionally after the surgery, while the treatment apparatus is on and providing the suction effect corresponding to the configuration.

### PRIORITY

This application claims priority of U.S. provisional application No. 62/275,813 filed on Jan. 7, 2016 and the contents of which is incorporated herein by reference.

### TECHNICAL FIELD

The present invention generally relates to medical methods. Especially, however not exclusively, the invention pertains to enhancing postoperative, preferably after an orthopedic or a plastic surgery, healing utilizing an apparatus producing low pressure suction, i.e. negative pressure flow, on a target area of a patient.

### BACKGROUND

Perioperative period refers to the time period describing the duration of a patient's surgical procedure. Perioperative period is commonly divided into three separate time periods. These include preoperative, intraoperative and postoperative periods. During the perioperative period, the goal is to provide better conditions for patient's recovery before, during and after the operation.

Common preoperative procedures include restraining from eating and/or drinking during a predetermined period, for example eight hours, before the procedure. Typically patients are prescribed to take blood thinning medicine such as aspirin to prevent clotting. Other medication may also be prescribed depending on the procedure and health of the patient.

Intraoperative procedures typically include anesthesia, monitoring the condition of the patient, and the actual surgical operation to be performed.

Postoperative period begins when the patient is transferred after surgery to the premises where the recovery from the procedure begins. The condition of the patient is monitored at least as long as the effect of anesthesia wears off. This may take, for example, one to two hours. Depending on the surgery performed and patient's overall health and condition, the patient may have to stay in the hospital for several days, even weeks.

The purpose of the care during preoperative period is to have a better surgical outcome, e.g., decrease the risk for complications, alleviate pain, reduce inflammation, and lessen the reduction of mobility due to the surgical procedure. Measures taken during the preoperative period are very important especially prior to any invasive procedure.

Typical postoperative procedures may include, for example in case of a knee surgery, compression boots that wrap around the lower leg to apply gentle pressure. Another method is using continuous passive motion (CPM) machine that gently moves the leg while patient rests in bed. Patient can also perform movement of the leg by himself. It is also beneficial to keep the limb in elevated position every day for a certain period of time in order to reduce swelling. Postoperative procedures also commonly include use of medication such as use of blood thinning medicines.

Postoperative procedures also include guided exercises and/or instructions for physical therapy including movements and exercises to be performed by the patient to help to adjust, for example in case of a knee surgery, to new joint and gradually begin to walk.

It is typical for the patients to experience pain, bruises and swelling after any surgery. These usually subside after a few weeks of rest and physical therapy. Swelling may be managed by the use of ice packs, for instance. The pain is typically treated with medication, if necessary.

Medication often has side effects as is well-known. The side effects can become worse in case the medication is continued to be used for a long period of time. In case of stronger pain relievers, such as opioids, the patient may develop an addiction to the medicine. In some cases new medication is prescribed to alleviate the side effects of the earlier medicine which as a combination can cause new symptoms. The surgical wounds must also be kept clean or otherwise they can easily become infected. Measures should be taken in order to initiate recovery as soon as possible in order to minimize the time period of patient lying still in the hospital bed and of medicine usage.

Lymphatic drainage is one option which can benefit patient recovering from a surgery. In lymphatic drainage, the lymphatic system is activated. Lymphatic system includes such elements as lymphatic vessels that carry fluid called lymph, lymph nodes, associated organs, such as the spleen and the thymus. Lymphatic drainage is naturally done when muscles contract and relax, thus moving the lymph in the lymphatic vessels. The system itself does not have ‘a pump’ such as the heart is for the blood circulatory system. If the patient doesn't move or is unable to move a lot, for example after a surgery, the waste from the tissue can accumulate in the lymphatic vessels and nodes, which causes swelling due to blockages. This is especially relevant when the lymphatic system is temporarily or permanently damaged and does not work properly.

Surgery poses a risk to compromise the lymphatic system when cut is made through lymphatic vessels and can cause lymphedema or inflammation. Typically it takes a few weeks to vessels to regenerate. Lymphatic drainage during the preoperative period can clear the lymphatic system by activating it and reduce chances of infections. Other benefits include increased blood flow, bringing nutrients to the tissues, reducing stress and anxiety. Lymphatic drainage during postoperative period activates the lymph flow when the patient otherwise lacks physical movement due to the surgery, it can reroute lymph to healthy vessels thus reducing swelling, bruises and minimizing scar tissue. It can also enhance skin tone and quality of the skin on the area on which the surgery has been performed.

Lymphatic drainage can be done manually by gently massaging the skin tissue in order to active the lymphatic system, i.e. manual lymphatic drainage (MLD). Therapists use rhythmic circular movements and only a specific low amount of pressure when massaging to stimulate lymphatic flow. Manual lymphatic massage requires typically a massage therapist to perform the procedure. Self-massage is also possible, however, can be exhaustive for an inexperienced person. Lymphatic drainage massage differs from traditional massage which uses more pressure applied to muscles rather than the gentle circular motion in order to move the skin tissue as in lymphatic drainage massage. This requires expertise to be performed properly.

Lymphatic drainage can also be done by using surgery such as liposuction, removal of abnormal lymphatic tissue and transplanting normal lymphatic tissue to areas with abnormal lymphatic drainage. Surgical means of lymphatic drainage naturally poses same risks as any surgery. Abovementioned methods apply mainly in the case of existing abnormal lymphatic tissues and are not preferable during perioperative period of a surgery in case of a patient with properly functioning lymphatic system.

### SUMMARY

The objective is to provide a method for enhancing postoperative, preferably after an orthopedic or a plastic surgery, healing using a low pressure suction apparatus so as to provide at least temporary relieve from the symptoms due to the surgery and to enhance the recovery from the surgical procedure while still preferably alleviating or overcoming one or more problems of the various prior art solutions.

Therefore, according to an embodiment, a method for enhancing postoperative healing using a low pressure suction apparatus comprising
obtaining a preferably both electrically powered and portable treatment apparatus capable of contacting, via a treatment head, a target area of a patient and introducing low-pressure suction, i.e. negative pressure flow, thereto,
configuring the treatment apparatus so as to introduce predefined, advantageously, pulsating pressure variation to a suction opening arranged at the treatment head, and
applying the suction opening of the treatment head on the target area before the surgery, i.e. during the preoperative period, while the treatment apparatus is on and providing the suction effect corresponding to the configuration.

The present invention is also, according to an embodiment, a method for enhancing postoperative healing using a low pressure suction apparatus comprising
obtaining a preferably both electrically powered and portable treatment apparatus capable of contacting, via a treatment head, a target area of a patient and introducing low-pressure suction, i.e. negative pressure flow, thereto,
configuring the treatment apparatus so as to introduce predefined, advantageously, pulsating pressure variation to a suction opening arranged at the treatment head, and
applying the suction opening of the treatment head on the target area after the surgery, i.e. during the postoperative period, while the treatment apparatus is on and providing the suction effect corresponding to the configuration.

The present invention is also, according to another embodiment, a method for enhancing postoperative healing using a low pressure suction apparatus comprising
obtaining a preferably both electrically powered and portable treatment apparatus capable of contacting, via a treatment head, a target area of a patient and introducing low-pressure suction, i.e. negative pressure flow, thereto,
configuring the treatment apparatus so as to introduce predefined, advantageously, pulsating pressure variation to a suction opening arranged at the treatment head, and
applying the suction opening of the treatment head on the target area before and after the surgery, i.e. during the preoperative and postoperative periods, while the treatment apparatus is on and providing the suction effect corresponding to the configuration.

In various embodiments, said configuring may include determination of at least one feature selected from the group consisting of: size of the opening at the treatment head, shape and/or dimensions of a treatment cup located at the treatment head, used treatment cup among multiple options of different material, shape, opening and/or dimensions, pulse duration, duty cycle, signal period, signal (repetition) frequency, pulse pressure, minimum, maximum or optimum duration of subjecting the treatment head to an area at a time, and minimum, maximum, or optimum duration of a treatment session. The beginning or end of any aforesaid duration may be optionally indicated audibly, tactilely (e.g. vibration) and/or visually to the patient or other operator of the apparatus during use for guidance.

In various embodiments, the apparatus may incorporate a central unit and a functionally connected treatment head that is preferably configured as hand-held by the patient or other operator of the apparatus. The connection may be established via a hose between the unit and head. The internal wall of said hose may define an air duct between a pressure chamber of the central unit and opening of the treatment head preferably provided by a treatment cup of the head. Further, an electrical connection between the two may be established via electrical wiring or wirelessly e.g. via electromagnetic coupling. The hose may transport gaseous matter, typically air, and thus cause the pressure pulsation due to a pulsating pumping action executed by pumping mechanism of the central unit. The central unit may include a vacuum pump for the purpose. In some embodiments, a fan could be alternatively utilized.

In various embodiments, said applying incorporates maintaining the portion, such as a cup, of the treatment head defining the opening substantially in contact, typically skin contact, with location at least partly defining the target area for a predefined time period, which may optionally refer to duration of few seconds, or e.g. about 3-5 pulsations.

Thereafter, the head may be re-positioned to a new location that may optionally overlap with the previous one. Such procedure may be continued until the target area as a whole has been treated at least once. This may include, preferably, at least three different spots on the target area, e.g. three to five different spots. In some embodiments, the treatment may be alternately directed to the same locations constituting the target area, e.g. as a repeated treatment pattern of several (sub-)areas with potentially overlapping portions, until a predefined overall period set for a treatment session, for instance, has lapsed.

The application technique in terms of motion may include stationary treatment. In stationary treatment, a certain target area or location, i.e. ‘sub-area’ therewithin in case the overall target area is too large for treatment by the treating head at a time (very typical scenario), is subjected to the low pressure suction treatment at a time by maintaining the treatment head thereon for some time, e.g. the aforementioned period of few seconds or few pulsations, prior to switching over to a next location. The switch over thus involves lifting the treatment head first away from the skin contact prior to moving it.

Alternatively or additionally, a so-called lift & twist-type technique may be utilized. While a certain location is treated and the treatment head is lifted from the skin, simultaneous rotating, or ‘twisting’, action is performed.

Alternatively or additionally, substantially continuous sliding type treatment technique may be applied by moving the treatment head over the locations of the target area while maintaining the contact of the treatment head. As one variation, ‘knitting’ style sliding with a twist motion may be tried.

As a further option, a hybrid approach may be selected implying keeping the treatment head stationary relative to one location accommodating the treatment head while during the switchover to a next location the contact is still maintained instead of lifting the head away. As being mentioned above, the subsequent location may overlap with the previous one.

In various embodiments where switchover between the locations of stationary treatment occurs, at least the two subsequent areas may have some overlap, e.g. about 20-50% overlap.

The utility of the present invention arises from a variety of factors depending on each particular embodiment thereof. The generated pulsation generally stretches and generally mobilizes the skin, therefore stretching fascia and affecting the related structures of myofascia (i.e. soft tissue manipulation), thus typically making additional clearance below it and reducing the related pressure subjected to tissues, organs, veins, lymphatic vessels, etc., while further activating them and e.g. the lymphatic system in general. The treatment may also yield various other advantageous effects described herein. These may include reduction of pain in patients and, thus, even facilitate reduction in pain medication use. In addition to negative/low pressure, i.e. suction, the pulsation may introduce certain amount of positive pressure to the tissues considering e.g. the areas opposite or adjacent to the target area under suction, or even to the target area itself due to the undulating nature of the pulsation and resulting skin motion. Accordingly, some benefits of positive pressure may be realized as well.

The treatment in the preoperative period may include, for example, one to three therapy sessions, duration of one session being about 10-60 minutes, preferably about 10-30 minutes, in some cases 10-20 minutes. Depending on the medical history, health and condition of the patient, treatment in the postoperative period may require at least one to two, in some cases five to ten sessions, duration of one session being 10-60 minutes, preferably 10-30 minutes, in some cases 10-20 minutes.

It should always be remembered that during the postoperative period open wounds are not meant to be treated directly and, e.g. in case of fresh scars (e.g. about two weeks after removing surgical sutures), the risk of damaging the tissue should be carefully evaluated before applying the low pressure suction directly on the target area, and the parameters of the treatment apparatus, such as the magnitude of the low pressure utilized, should be adjusted accordingly.

With the suggested therapeutic procedures, the swelling, puffiness and bruising associated with a surgical procedure may be reduced, the drainage of the lymph may be enhanced, pain may be relieved, skin elasticity increased and general condition of the patient may improve. Advantageously, in addition to pulsating or continuous low pressure suction, a high frequency oscillation treatment may be utilized in order to reduce swelling of the area to which the surgical procedure has been performed. If the higher frequency oscillation is used, higher pressure values of the negative pressure may preferably be used.

The embodiments of the present invention are considered both non-invasive and safe. The apparatus or generally equipment used to execute the treatment is affordable, portable, reliable, quiet and easy to service or use either by a patient or separate operator. The user, either being the patient himself/herself or dedicated operator, is not required to take extensive training to be able to apply the apparatus although at least basic understanding of human lymphatic system and operation of the apparatus is naturally considered advantageous in favor of the effectiveness of the therapy.

Accordingly, the therapy may be provided flexibly at different premises such as the home of a patient, at a physical therapist, or at some other desired location. There is no need to visit a doctor, hospital or some specialized therapy center to receive the treatment, which may facilitate the life of the patient considerably in terms of reduced travelling and associated cost, and gained time savings among other factors.

Finally, use of the apparatus is practically completely painless as the pulsation pressure may be adjusted to suit each use scenario such that the effect of the treatment is achieved while the suction effect remains moderate only causing gentle draft and pull type stretch sensations on the skin in contrast to different prior art methods and apparatuses, the effect of which is at least partly based on harsh, mechanical skin stretching and actually pinching activity due to the use of gripping elements such as rollers, which may be rather painful on sensitive swollen and potentially infected skin.

Relying on an apparatus according to an embodiment of the present invention may in many cases turn out advantageous also to the operator in contrast to e.g. manual therapy. The elbows, wrists, and hands of the operator may be spared from fatigue and pain, which are commonly induced by lengthy days of manual therapy.

Various other advantages will become clear to a skilled person based on the following detailed description.

The expression “a number of” refers herein to any positive integer starting from one (1), e.g. to one, two, or three.

The expression “a plurality of” refers herein to any positive integer starting from two (2), e.g. to two, three, or four.

The terms “first” and “second” do not denote any order, quantity, or importance, but rather are used to distinguish one element from another.

The exemplary embodiments of the invention presented in this patent application are not to be interpreted to pose limitations to the applicability of the appended claims. The verb “to comprise” is used in this patent application as an open limitation that does not exclude the existence of also un-recited features. The features recited in depending claims are mutually freely combinable unless otherwise explicitly stated.

The novel features which are considered as characteristic of the invention are set forth in particular in the appended claims. The invention itself, however, both as to its construction and its method of operation, together with additional objectives and advantages thereof, will be best understood from the following description of specific embodiments when read in connection with the accompanying drawings.

### BRIEF DESCRIPTION OF FIGURES

The embodiments of the invention are illustrated by way of example, and not by way of limitation, in the figures of the accompanying drawings.

FIG. 1 illustrates schematically an embodiment of a treatment apparatus in accordance with the present invention.

FIG. 2 illustrates schematically an embodiment of a treatment apparatus in accordance with the present invention.

FIGS. 3A and 3B illustrate embodiments of a treatment cup of the treatment head in connection with the treatment apparatus in accordance with the present invention.

FIGS. 4A and 4B illustrate embodiments of a sealing part of the treatment head in accordance with the present invention.

FIG. 5 depicts a use scenario of low pressure suction apparatus in treating the leg of a patient before the surgery, i.e. during the preoperative period.

FIG. 6 depicts a use scenario of low pressure suction apparatus in treating the leg of a patient after the surgery, i.e. during the postoperative period.

FIG. 7 is a flow diagram disclosing an embodiment of a method in accordance with the present invention.

### DESCRIPTION OF SOME EMBODIMENTS

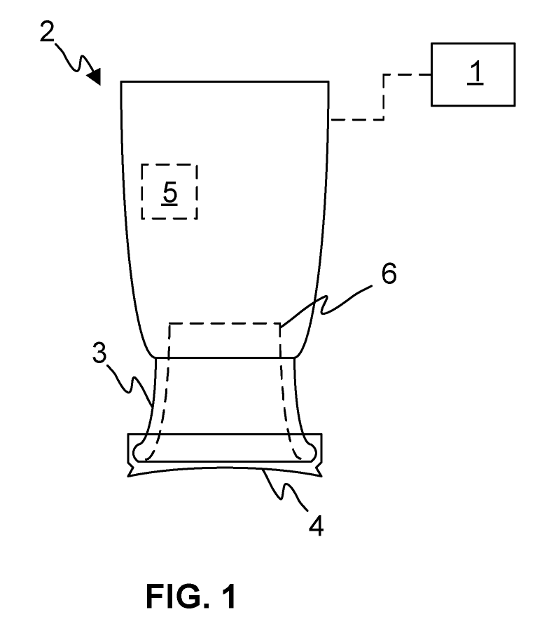

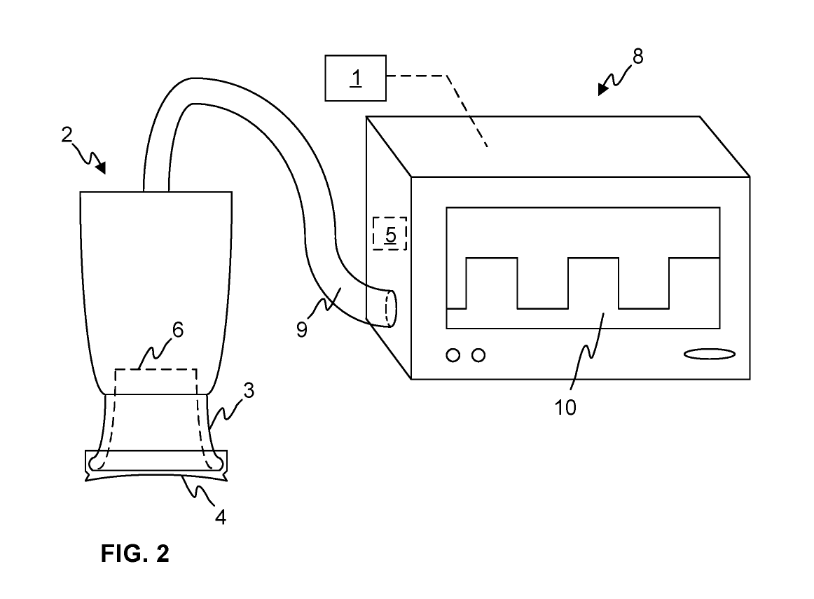

With reference to FIGS. 1 and 2, two embodiments of a treatment apparatus, or ‘device’, for use in connection with a therapeutic method in accordance with the present invention are illustrated.

Embodiments of the treatment apparatus comprise a treatment head 2, a central processing unit 1, a treatment cup 3, a sealing part 4, low pressure chamber 6 and means for producing the low pressure in the low pressure chamber 5. The central processing unit 1 may be arranged in connection with the treatment head 2 or in connection with a central unit 8 as shown in FIG. 2. The low pressure chamber 6 and the means for producing low pressure in the low pressure chamber 5 are shown schematically by dashed lines in FIGS. 1 and 2. The means for producing the low pressure 5 (negative pressure flow/suction effect), e.g. with a vacuum pump, may be in the treatment head 2 or in the central unit 8 in which case the means for producing the low pressure 5 is arranged in connection with the treatment head 2, e.g., by a hose 9. There may be a display in the treatment head 2 and/or 10 in the central unit 8. The central unit 8 may further incorporate an interface for an external display. In some embodiments, the features of a central unit 8 could even be combined with the treatment head 2 into an integral apparatus. In some embodiments, the pump or fan 5 could be included in the treatment head 2 while there is still provided a central unit 8 or similar element connected to the head 2 by a flexible element such as a cable or wiring for electricity and/or data transfer, for example. These types of embodiments would not obviously necessitate the use of a hose 9 for the suction flow between the elements 2, 8. When the pump or fan 5 is located within a common housing with the chamber 6, these two may still be connected via internal piping, hose(s) and/or duct(s), for example, for conveying the low pressure flow.

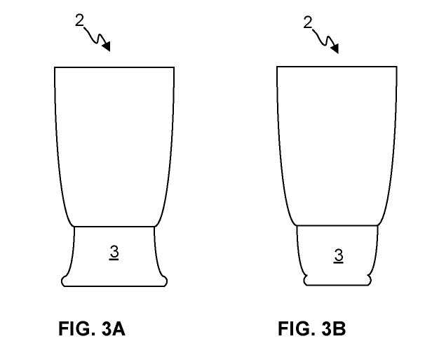

FIGS. 3A and 3B illustrate two embodiments of the treatment cup 3, one end of which is arranged to be in connection with the low pressure chamber 6 and the other end of which is arranged to be pressed towards (meaning in this application a degree of contact or a magnitude of force from a gentle touch/contact to more intense pressing) the skin tissue to direct low pressure suction to the target area, typically skin tissue, thus producing a bulge in the skin tissue. The skin preferably refers herein to all skin layers i.e. epidermis, dermis, hypodermis or subcutis.

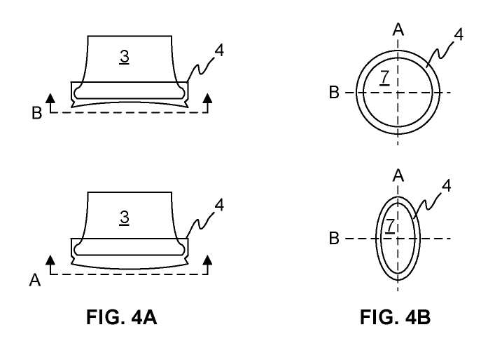

In FIGS. 3A and 3B, the end of the treatment cup 3 that is arranged to be pressed towards (typically against) the skin tissue is the lower end of the treatment cup 3. The treatment cup 3 is arranged to form a suction opening 7 (shown in FIG. 4B). FIGS. 3A and 3B illustrates two different shapes of the treatment cup. There may also be other shapes and, thus, the examples in FIGS. 3A and 3B are not shown to pose limitations to the treatment apparatus utilizable in connection with the method according to the present invention. The treatment cup 3 with an appropriate size, shape and/or material may be chosen based on the nature or size of the target area of the skin to be treated, for instance.

The treatment cup 3 may further comprise a sealing part 4 advantageously made of a flexible material and arranged at the end of the treatment cup 3 that is arranged to be pressed against the skin tissue and to adapt to the shape of the suction opening 7 formed by the treatment cup 3 and to seal the gap between the end of the treatment cup 3 and the skin tissue when the end of the treatment cup 3 is pressed against the skin tissue.

The sealing part 4 may be made of any suitable flexible material, such as polyurethane or elastomeric material. The use of other plastic materials and silicon material is also possible. At the sealing part 4, it is naturally also possible to use a suitable material that reduces or increases the friction between the sealing part 4 and the skin tissue, and/or a material that improves the sealing, depending on whether the objective is to achieve a good mobility for the treatment head or as high a friction force as possible. Mobility may also be increased by using a number rotating elements, such as rollers, which are in connection with the treatment cup 3.

The rotating elements, in addition to the sealing part 4, may also provide sealing function between the skin and the treatment cup 3.

As the treatment head 2 of the treatment apparatus is moved against the patient's skin, most comfortably using a handle, the effect of the low pressure causes a fold of skin to be pulled up into the low pressure chamber 6.

FIGS. 4A and 4B illustrate the shape of the sealing part 4 by side views from two substantially perpendicular directions. The sealing part 4, which is to be pressed against the skin tissue, is arranged to be concave in the direction of at least one of the main axes A or B of the suction opening 7 formed by the treatment cup 3.

In an embodiment of the treatment cup 3 of the treatment apparatus shown in FIG. 4A, the surface of the sealing part 4 to be pressed against the skin tissue is concave in the direction of the main axis B of the suction opening 7 formed by the treatment cup 3. The shape of the suction opening 7 may be elliptical or oval or round (some examples are shown in FIG. 4B). However, these are not the only feasible embodiments but other shapes may be used as well.

In the above examples, the sealing part 4 is shown as a separate part fastened to the end of the treatment cup 3. The sealing part 4 may be a disposable part, which is detached after use and replaced by a new one in the beginning of the treatment of a new patient. Alternatively, the sealing part 4 may be reusable after washing or disinfecting. The fastening to the end of the treatment cup 3 may be achieved by means of various connections. At the end of the treatment cup 3, which is to be pressed towards (typically against) the patient's skin, there may be a flange in the radial direction, which is used for fastening the sealing part 4 made of a flexible material to its place. Thus, the schematically shown connection of the sealing part 4 in figures is not to be understood as a factor limiting the embodiments of the treatment apparatus used in connection with the method according to the present invention. It is also to be noted in this context that the sealing part or portion 4 may be formed as an integral part of the treatment cup 3, for instance.

In various embodiments, an opening for subjecting the target tissue such as skin to the suction may be defined by a treatment cup 3 (part or portion) of the treatment head 2. The treatment cup may be adaptable in size to best fit the shape of the treated body part. The adaptability may be implemented by a plurality of interchangeable cups of different size and/or by an adjustable cup. For example, the applicable size may range from about 10 or 20 mm to 80 or 90 mm in diameter depending on the dimensions and shape of the target area. As a general rule, a largest cup considered suitable for the area may be selected. For instance, 60-80 mm size may be more suitable for the back or neck than for facial areas that benefit from using a smaller diameter cup and related opening.

The treatment apparatus is thus not restricted to the examples of the figures in any way, but the apparatuses may be varied entirely freely within the scope of the claims. Thus, it is clear that the invention is by no means restricted to any specific shape or dimension of the treatment cup 3 or other components, for instance, but the shape and/or dimensions of the different elements and parts of the invention may differ from one another freely between embodiments, if desired. The idea of the present invention may even be applied in connection with such treatment heads at which rotating elements such as rollers are employed, as mentioned hereinbefore.

The low pressure in the low pressure chamber 6 is advantageously produced by using the aforementioned vacuum pump. Necessary adjusting valves are also advantageously mounted in connection with the vacuum pump.

In addition, in case an embodiment of the treatment apparatus with a central unit 8 is utilized, the hose 9 may have a valve which is advantageously positioned near the low pressure chamber 6. The speed of the system may further be improved by using the hose 9 as a low pressure reservoir. Thus, higher or lower pressure may be achieved in the hose 9 compared to the pressure desired in the low pressure chamber 6, especially in case a central unit 8 is utilized. If, for example, change of pressure from 150 to 200 mmHg is desired, the pressure of the low pressure chamber 6 is 150 mmHg and if in the subsequent phase a pressure of 200 mmHg is desired, the pressure of the hose 9 may already be set for example to 500 mmHg, so that upon opening of the valve a pressure of 200 mmHg is achieved quickly in the low pressure chamber 6 and the valve may be closed. The valve may be arranged to be controlled by pulse width modulation for adjusting the low pressure in the low pressure chamber 6. In some embodiments the valve may control higher frequency oscillation (described in more detail hereinafter) produced in addition to lower frequency suction pulses. Alternatively, higher frequency oscillation could be produced by other element(s), such as electric motor or Vibrator′. In some embodiments, the oscillation could be at least temporarily solely produced (i.e. no simultaneous lower frequency pulsation).

Subsequently, if a pressure of 150 mmHg is again desired, in which case a pressure of for example 50 mmHg may be set in the hose 9 so that a change from 200 mmHg to 150 mmHg is quick. Furthermore, the low pressure chamber 6 when positioned against the target area (skin) may be either substantially sealed or it may have a controlled leakage, for example, through a small opening.

The treatment apparatus utilizable in the method according to the present invention preferably comprises different sensors, one of which, for example, measures the composition of the skin tissue, such as the fluid content, fat content and/or oil content. Separate sensors may also be used for measuring fluid and fat contents. One sensor may, for example, measure the raised skin (bulge) produced by the suction effect and one other sensor may measure, for example, the suction force applied to the skin. The low pressure suction and suction force may be adjusted according to the results of the measurements. Therefore, the apparatus may be adaptive and especially dynamically adaptive. Yet, the treatment apparatus may comprise a sensor which measures the skin temperature.

Still, the apparatus may in some embodiments comprise a sensor for measuring the skin's blood circulation, the measurements of which may be used to adjust of the operation of the treatment apparatus. Further, the adjustment may be based on measurement of transepidermal water loss and skin pH.

Each sensor may be in connection with the treatment head 2 or to the treatment apparatus through a wired or wireless connection, such as, for example, a radio frequency signal, infrared signal or the like. Thus, the sensors may be an integrated or separate part of the treatment apparatus.

The apparatus may have at least one sensor that registers a signal given by the patient for increasing/decreasing the suction effect, based on which the adjustment of the suction may be done. The patient may thus give a signal to the sensor (for example, based on the pain experienced) and the sensor then relays to the apparatus the wish for the increase/decrease of suction efficiency. The sensor, such as a touch-registering sensor (e.g. a button), may be included in a user interface of the apparatus.

In one embodiment of the treatment apparatus, it may further comprise additional energy source(s) for warming the skin tissue and furthermore, means for automatically adjusting the energy source(s) to a set point value based on the measurements obtained by one or more sensors. Energy source may also be utilized to power up the apparatus for treating a patient.

Measurement techniques utilized in the embodiments of the treatment apparatus may include measurement of different sound frequencies, such as ultrasound and infrasound, techniques based on radiofrequencies and different wavelengths of light, i.e. optical measurement such as laser and infrared measurement, bioimpedance spectroscopy, magnetic resonance spectroscopy, Raman spectroscopy, nuclear magnetic resonance spectroscopy, microsensor mapping, heat camera imaging, or spectrofotometric intracutaneous imaging.

In some embodiments of the treatment apparatus, the potentially automated adjustment of the low pressure suction and/or suction force is based on mechanical characteristics and/or electrical characteristics and/or structure and/or composition of the skin. Mechanical characteristics include strength, flexibility, elasticity and resilience etc. Electrical characteristics include, for example, capacitance, impedance, resistance, reactance and inductance.

In addition, the potentially automated adjustment of the apparatus may in one embodiment be based on measurements of the flow of lymphatic fluid. The associated measuring techniques for the flow of lymphatic fluid may be selected from known techniques, such as, but not limiting to, isotope clearance technique.

In one embodiment of the treatment apparatus, the adjustment may also be based on the measurement of the patient's experience of cutaneous pain. Based on the experience, either the patient him/herself, or the operator, or both together adjust the apparatus's running parameters. Skin characteristics, when mentioned in this text, also include the pain felt and experienced on the skin.

In an embodiment of the treatment apparatus, a computer program guides in the application of the suction force by presenting the force level audibly and/or visually in the treatment head 2 and/or in the central unit 8 (e.g. via display 10 in case of visual information). Low pressure suction may be adjusted automatically using the computer program, and thus it is not necessary for the patient or operator to adjust the low pressure suction during the treatment. Advantageously, when the treatment force exceeds the permitted value, the program stops the apparatus or lowers the suction force.

The computer program may be utilized to calculate the target value of one or more on-going treatment forces, such as suction force, based on the measurements obtained and/or on the desired value of the suction pressure. Therefore, the apparatus also comprises sensor/sensors as mentioned hereinbefore for measuring one or more ongoing treatment forces, such as the level of the suction force. A computer program may be configured to automatically calculate and adjust the level of low pressure suction to the target value, based on the measurements obtained. The parameters/results of the measurements, which may be taken into account in determining the target value of the low pressure suction, include e.g. fluid content of the skin tissue, fat content of the skin tissue, the bulge i.e. the lift of the skin tissue (the size of the fold in the skin) and/or the skin temperature.

Furthermore, the computer program may be functionally connected to database, which contains the patient's treatment information. The database may be remote and hosted by a remote computer or computer system, which is accessed via a communications connection or network, e.g. the Internet. For the purpose the treatment may include a wired or wireless data interface, e.g. USB (Universal Serial Bus), Bluetooth™, NFC/RFID (Near-Field Communication/Radio Frequency Identification), cellular, wireless LAN (Local Area network) or wired LAN interface.

In one embodiment of the treatment apparatus, it is desired to combine slow, pulsating low pressure to a faster impulse-like oscillation treatment. For instance, the oscillation treatment may be modulated by a pulsating low pressure treatment.

In one embodiment of the treatment apparatus, the treatment apparatus is arranged to provide to the low pressure chamber 6 simultaneously a pulsating low pressure treatment, which preferably has a frequency of below 5 Hz, and an oscillation treatment, which for its part, preferably has a frequency of more than 5 Hz. In other embodiment, the threshold frequency between low pressure pulses and high frequency oscillation may be different.

Moreover, a high frequency oscillation treatment may be particularly added to the suction phase of the low frequency pulsating low pressure treatment. The oscillation may extend over the whole duration of the low pressure pulse. In some embodiments, in addition to the duration of the pulse, the oscillation could be present also during the neutral portion of the signal period. In some embodiments, the duty cycle of 100% (i.e. continuous suction/pulse) may be applied, whereupon also the optional oscillation may be on all the time. High frequency in this instance may mean for example an impulse-like pressure change or oscillation with a frequency of more than 5, 10, 15 Hz or higher frequencies such as e.g. 90 Hz or even as high as 200 Hz. The hose 9 may be arranged in this case to be used as a pressure reservoir for accelerating pressure variations in the low pressure chamber 6.

When using the high frequency oscillation, higher frequencies affect the (skin) tissue closer to the surface of the skin while with lower frequencies the effect of the treatment ‘penetrates’ deeper into the (skin) tissue. Thus by varying the frequency of the high frequency oscillation, the depth of the effect of the treatment may be varied and problems at different depths of the tissue may be more effectively treated.

In an embodiment, the pulsation frequency may range from about 0.1 Hz to about 5 Hz, or occasionally even up to 10 Hz depending on the particular embodiment of the apparatus (supported frequencies). For instance, it may be about 0.5 Hz that corresponds to a 2 second signal period and 1 second pulse duration with 50/50 duty cycle. The suction on the skin tissue when the treatment cup 3 is facing the skin is naturally introduced during the associated on-time (i.e. 1 second in case of 2 second total signal period with 50/50 duty cycle).

Preferably the configured frequency is user-adjustable via a user interface of the apparatus. The user interface may include a number of control input elements in the form of a touch display, touch pad, button, mouse, Scrollpoint™, roller, voice input interface, keypad, etc. for the purpose.

Accordingly, the UI (user interface) may include e.g. a display and/or audio response interface (typically buzzer or loudspeaker) for data visualization and feedback provision towards the apparatus operator. The operator may be the patient himself/herself or other person who preferably has adequate medical and technical skills to operate the apparatus in sufficient fashion. Typical pressure (suction, i.e. negative pressure) of the treatment apparatus may preferably be of the order of about 80 mmHg, falling e.g. within a range from about 30 to about 250 mmHg, or even up to about 350 mmHg or higher, e.g. about 500 mmHg. As being clear based on this disclosure, the negative pressure is preferably user-adjustable or -selectable in at least most embodiments. However, use of fixed (user non-adjustable) pressure and potentially other fixed parameters is possible in some embodiments of the apparatus as well. Such embodiments could be targeted to certain very specific use scenarios or applications, for example.

Typically higher pressure values are used in conjunction with the high frequency oscillation compared to the case with only low frequency pulsating treatment. Pressure values in the above mentioned cases may be, e.g., 100-130 mmHg and 50-80 mmHg, respectively.

In some embodiments, the operator of the device is provided, via the UI of the device, a pressure setting and/or pressure readings in predetermined, optionally user-selectable, units such as mmHg or pascal. Alternatively or additionally, e.g. a numeric value in a predetermined scale, e.g. between one and five or one and ten, without any particular units could be used for adjustments and/or indicated to the operator. One end of the scale could represent predetermined minimum suction or zero suction, whereas the other end (e.g. maximum number) could represent predetermined maximum suction. The apparatus manages the conversion between the user-indicated pressure and corresponding real pressure established.

Instead or in addition to numeric values, the pressure and/or other parameters could be indicated through other symbols, optionally using dot/circle, star, line, curve or rectangular shapes.

The order of magnitude of high frequency oscillation may range, for example, from about 2 to about 200 Hz, preferably at least from about 5 or 10 Hz to e.g. about 100 Hz.

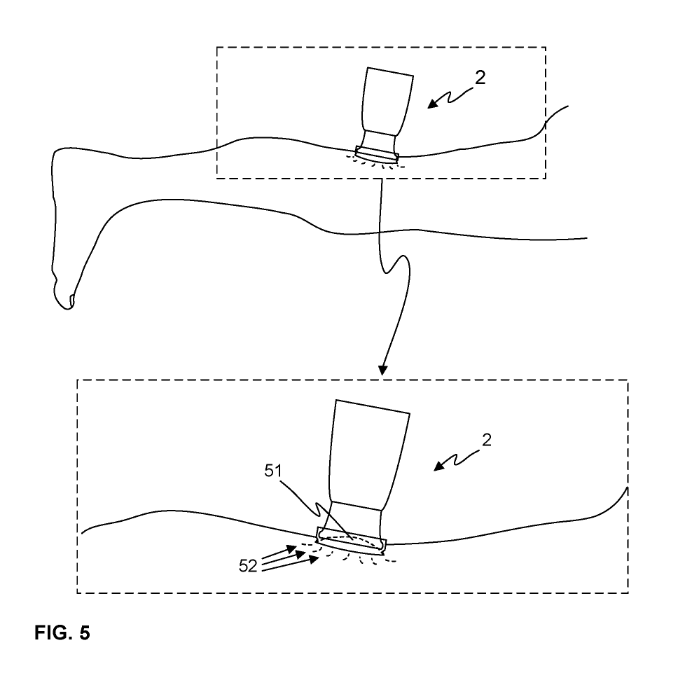

FIG. 5 depicts a use scenario of low pressure suction apparatus in treating, in particular, the target area of patient's leg during the preoperative period of the perioperative period, in this case, of a knee surgery. The patient may sit or stand in upright position during the treatment or lie down facing up or down, for instance. The treatment head 2 of the treatment apparatus may also be applied from below the leg while the patient is sitting or lying down facing up. The suction may be applied to the ‘knee pit’, i.e. popliteal fossa, at which the lymphatic vessels are closely located to the skin. The suction may also be applied other areas of the knee. The effect of the low pressure suction on the skin tissue is illustrated in FIG. 5. The suction causes ‘a bulge’ 51 on the skin, lifting part of the tissue into the treatment head 2. The suction simultaneously causes skin to stretch in other areas around the area on which the suction opening of the treatment head 2 is applied. This is shown in the figure with short curved lines 52. The stretching of the skin is not necessarily confined to the area of the treatment head and the immediate area around it. The suction may also, for example, cause stretching of the skin, e.g. on the skin at the sides of the knee or on the area opposite to the ‘kneepit’.

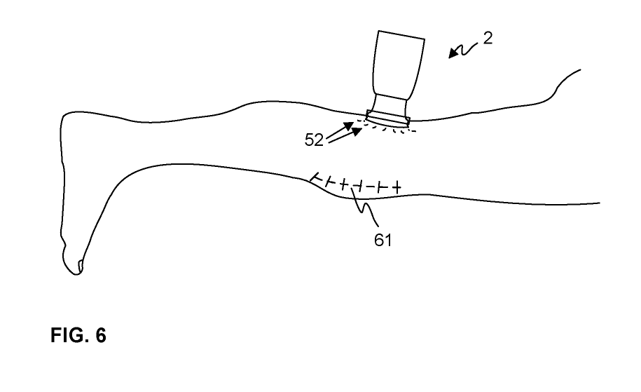

FIG. 6 depicts a use scenario of low pressure suction apparatus in treating, in particular, target area on patient's leg during the postoperative period of the perioperative period, in this case, of a knee surgery. The patient may sit or stand in upright position during the treatment or lie down facing up or down, for instance. The treatment head of the treatment apparatus may also be applied from below the leg while the patient is sitting or lying down facing up. The suction may be applied to the ‘knee pit’, i.e. popliteal fossa, at which the lymphatic vessels are closely located to the skin. The suction may also be applied other areas of the knee. The suction simultaneously causes skin to stretch in other areas around the area on which the suction opening of the treatment head is applied. The suction may also cause stretching of the skin, e.g. on the skin at the sides of the knee or on the area opposite to the ‘kneepit’. The stretching of the skin is not necessarily confined to the area of the treatment head and the immediate area around it. The parameters should be configured such that the surgical wound 61 is not stretched too much in order to avoid, e.g. removal of stiches.

Optionally, lubricant such as massage oil may be initially smeared on a target area of the skin. Care shall be taken that the lubricant does not contain particles that could end up within the apparatus during the treatment to avoid clogging the internals thereof and related cleaning procedures.

The treatment head is located so that the associated contact portion, such as preferably replaceable treatment cup, is in close contact with the skin area to be treated, e.g. on the popliteal fossa (shown in FIGS. 5 and 6), trapezius muscle, spine, or e.g. sternum.

The diameter of an optimum cup generally varies between patients and from a treating technique to another, but in standard case it may range from about 60 mm to about 80 mm, for example. As an applicable basic rule, one could consider to select the largest suitable treating cup for each target area. Fastening of the treatment cup having regard to the rest of the treatment head may incorporate grooves (in the cup or head) and matching lips (in the head or cup, respectively), snap fastener(s), threads, magnets, frictional and/or pressure contact (e.g. based on the elasticity (enabling stretching) and/or roughness of the contacting surface(s) yielding tight, secure fit), or any combination of the above or other feasible attachment technologies providing e.g. sufficiently secure and airtight fit between the connected elements.

The treatment head/cup should be hold onto relatively lightly. The cup may typically be kept on the same location for about three to five pulsations, whereafter it may be moved to adjacent skin area with e.g. ⅓ overlap. The total number of locations, or spots, that are treated depends on the overall coverage of the treated condition, which typically defines the target area of treatment, as well as the size of the cup and related suction opening. The area may encompass from about one or two to ten locations, for instance.

Either stationary, sliding or hybrid technique may be applied having regard to the lifting of the treatment head during the movement thereof on the skin between the different treated areas. The general direction of motion may be sideways and/or from the top to the bottom (i.e. from the head towards the torso or shoulder line). Thus the overall duration of treating a certain area or spot at a time commonly ranges from about few seconds to few tens of seconds depending on the utilized pulse duration and duty cycle and thus the overall signal period.

Pulse length/duration may be about one second, for example, and the used pressure between 5-350 mmHg, advantageously from about 20 mmHg to about 130 mmHg. Duty cycle may be about 50/50 (50%) between the pulse period and passive period, i.e. pulse-containing and neutral portions of the treatment signal, respectively, whereupon the overall repeating signal period comprising the pulse portion and neutral/passive portion covers two seconds. The duty cycle with two-second signal period may also be, e.g. 20/80 (20%) or 30/70 (30%) in which cases the pulse-containing portion lasts 0.4 or 0.6 seconds and neutral portion 1.6 or 1.4 seconds, respectively. The signal period may be varied preferably from a half to five seconds thus meaning pulsation frequency of 0.2 to 2 Hz. The high frequency oscillation additionally provided at least or exclusively during the pulse-containing period (1 second in case of duty cycle of 50/50 (50%) in case of a two-second signal period) may preferably be from 20-90 Hz.

One shall acknowledge the fact that also the neutral period (at least no low frequency suction) is usually treatment-wise important e.g. in a sense that during it the skin stretching stops and the skin recovers its relaxed position. The effectiveness of the treatment is in many respects due to the back-and-forth movement of the skin, not just due to suction-based stretching thereof.

Having regard to various embodiments, typically one therapy session lasts for about 10-60 minutes, preferably about 10-30 minutes, at a time. A treatment period may include multiple sessions, e.g. at least one or about three to five sessions during the preoperative period and at least one or two, or in some cases about five to ten sessions during the postoperative period. As mentioned hereinbefore, in many cases the obtained results are not necessarily permanent, whereupon the therapy should be regularly practiced even after a more intensive therapy period, e.g. once a week.

Considering the general direction of motion of the treatment head in the therapy sessions, one could conclude that typically the locations closer to the two subclavian veins may be treated prior to locations farther away therefrom, e.g. limb extremes, so that the main lymphatic channels are opened for the lymphatic flow first instead of trying to open the extreme conduits first while the flow is still blocked centrally.

A preoperative as well as a postoperative therapy session performed on, e.g. right leg, may include first applying the treatment head in the area of supraclavicular fossa, close to subclavian veins, and, preferably, the axillary lymph area. Because the lymphatic system is unsymmetrical in a way that the supraclavicular area on the left side of the body takes care of roughly everything else except the areas of right arm and portion of the chest, draining the left side is especially important, although treating the lymphatic system on the right side is also important and may optionally be performed. Therefore, preferably, the left arm, being part of the larger lymphatic system of the left side of the body, should also be drained in case of, e.g. surgery on right leg. After that the lymphatic vessel in the chest and stomach and/or in the back are treated so as to proceed from the area of supraclavicular area towards the area on which the surgery will be performed. Along the way, the pelvic lymph nodes, especially on the right side in case of surgery to right leg, are opened. Then the treatment continues to the area on which the surgery is to be performed, i.e. to the right leg in this case. The area of the surgery should be more thoroughly treated in order to drain the lymphatic vessels properly, thus removing inflammatory agents, large molecules such as proteins which tend to absorb fluids. Typical postoperative therapy session may be similar to the typical preoperative session with the exception that special attention must be given to area of the surgical wound, for example, not to apply excess suction too close to the wound so as to accidentally remove stitches or open the wound.

The activation of the lymphatic system may include the use of one or several different techniques such as stationary and sliding techniques. Especially, the stationary technique is used when proceeding towards the surgical area and the most peripheral parts of the lymphatic system, such as e.g. ankles. Sliding technique may be especially useful when moving from the most peripheral parts of the lymphatic system back towards the area of supraclavicular fossa, thus ‘pushing’ the lymph towards the area of the subclavian veins for collection and returning to bloodstream.

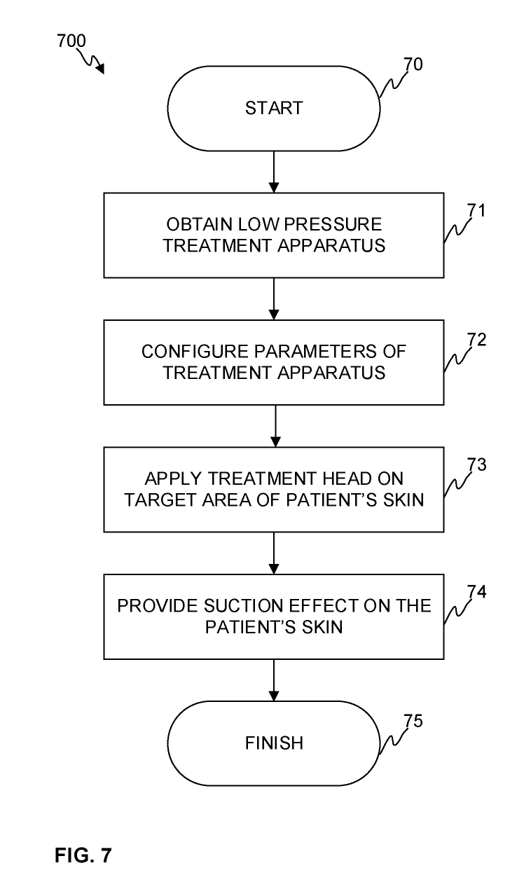

FIG. 7, at 700, is a flow diagram disclosing an embodiment of a method in accordance with the present invention.

Item 70 refers to a start-up phase. Decision to treat a patient in accordance with the principles of the present invention is made.

At 71, an embodiment of a treatment apparatus is obtained. It may be purchased, borrowed or rented, for example. Further gear, such as massage oil and treatment chair or table may optionally be further acquired at this stage.

At 72, the apparatus and possible other equipment (e.g. chair/table) are configured, which may refer to adjusting, via the UI of the apparatus, desired parameters for the treatment including e.g. suction pressure, pulsation frequency, oscillation frequency, duty cycle, etc. A patient may himself/herself configure and subsequently utilize the apparatus. Alternatively, the apparatus may be operated by some other party, such as a professional operator such as a medical professional, a therapist, a nurse, a friend or a family member of the patient. Already for the configuration tasks, the apparatus may have to be turned on unless the configuration can be purely adjusted by using e.g. mechanical switches, sliders or other elements that continuously remember their state in contrast to e.g. touch pads or touch displays that have to be powered up first to register the user input.

At 73, the treatment head is (re-)positioned on a target area or sub-area thereof to be treated, which usually involves placing the head, or in practice the contact part of the associated treatment cup or similar element, in contact with the skin of the patient. Depending on the nature of the treatment, stationary, sliding or hybrid application technique may be selected.

At 74, the treatment is executed having regard to the target area by subjecting the area to the low pressure (suction) pulses and intervening neutral or passive periods.

In practice, items 73 and 74 are usually simultaneously and/or sequentially repeatedly executed during a treatment session. Accordingly, their mutual execution order may be considered to vary. The treatment head/cup at a certain location usually covers only a small subarea of the overall target area at a time, whereupon the head shall be moved along a desired route on the skin to cover the target area in its entirety using a preferred application technique.

Method execution is ended at 75. The suction and the apparatus in general may be turned off. Alternatively, at least some measurement results and/or other gathered statistics concerning the treatment, e.g. pressure, pulse characteristics, or various durations, may be inspected via a display or other feasible element of the treatment apparatus itself, or outputted therefrom via an available data communication interface either wirelessly or wiredly. The data may be transferred to a near-by apparatus or a remote system, optionally via the Internet, for storage, inspection (e.g. visualization) and/or analysis. The external apparatus or system may host a database for storing data from a number of treatment apparatuses. The patient may be instructed to drink water to prevent dehydration.

In some variations of the treatment apparatus described herein, the treatment head and related elements, e.g. suction cups thereof, could be configured for enabling substantially contactless operation in addition to or instead of contact-based therapy. During the contactless therapy, the treatment head could be merely hovered close to the target area without actually contacting the skin, for instance. The apparatus could be provided with audible, tactile and/or visible guidance element such as loudspeaker, buzzer, vibration element, indicative lamps (e.g. LEDs) and/or a display, optionally touchscreen, The guidance element could indicate, in real-time fashion, current and/or proper distance between the target surface (e.g. skin) and the treatment head. In contact-based treatment, the cup/flexible element of the treatment head inherently provides such guidance for maintaining a proper distance (in that case, contact) between the head and target surface.

1. A method for enhancing postoperative healing using a low pressure suction apparatus comprising
obtaining a preferably both electrically powered and portable treatment apparatus capable of contacting, via a treatment head, a target area of a patient and introducing low-pressure suction, i.e. negative pressure flow, thereto,
configuring the treatment apparatus so as to introduce predefined, advantageously, pulsating pressure variation to a suction opening arranged at the treatment head, and
applying the suction opening of the treatment head on the target area before the surgery, i.e. during the preoperative period, while the treatment apparatus is on and providing the suction effect corresponding to the configuration.

2. The method of claim 1, spanning several treatment sessions distributed over time, optionally substantially regular sessions, or at least one session in the preoperative period, preferably of at least about 10 minutes in duration per session.

3. The method of claim 1, spanning several treatment sessions distributed over time, optionally substantially regular sessions or at least one session in the postoperative period, preferably of at least about 10 minutes in duration per session.

4. The method of claim 1, comprising applying the suction opening of the treatment head on the target area after the surgery, i.e. during the postoperative period, while the treatment apparatus is on and providing the suction effect corresponding to the configuration.

5. The method of claim 1, wherein the treatment head comprises a preferably replaceable treatment cup for contacting the skin of the patient, preferably without substantial clearance, further wherein a proper cup is selected from a plurality of cups responsive to the characteristics and/or location of the target area to be treated.

6. The method of claim 1, wherein the diameter of the suction opening of the head, preferably of the cup thereat, falls within a range of about 10 mm to about 85 mm.

7. The method of claim 1, wherein the pulsating pressure variation comprises low pressure pulses substantially in the range from about half a second to two seconds, preferably about one second, in duration and/or from about 5 mmHg to 350 mmHg, advantageously from about 20 mmHg to 130 mmHg in strength.

8. The method of claim 1, wherein an overall signal period that is then repeated contains a pulse period with suction effect and temporally adjacent passive period substantially introducing no suction, wherein the relationship between the two is optionally determined by a preferably user-adjustable duty cycle parameter of the apparatus.

9. The method of claim 1, wherein the pulsating pressure variation includes, in addition to low frequency pulses, simultaneous higher frequency oscillation, preferably falling within a range of about 5 to about 100 Hz.

10. The method of claim 1, wherein the treating head is maintained at a certain location of the target area for a duration covering about 3-5 pulses at a time.

11. The method of claim 1, wherein two successively treated locations of the target area overlap, preferably about 20-50%.

12. The method of claim 1, wherein a number of same locations are alternately treated during a single treatment session.

13. The method of claim 1, wherein the application technique of the head is, in terms of motion, stationary, sliding or a hybrid between the two involving maintaining the head at a certain location for some time while also sliding the head substantially in skin contact between locations.

14. The method of claim 1, wherein lubricant, optionally massage oil, is initially applied to the target area.

15. The method of claim 1, wherein the apparatus comprises a central unit connected to the treatment head at least by a hose.

16. An electrically powered, portable treatment apparatus for enhancing postoperative healing, wherein the apparatus comprises a treatment head configured to contact a target area of a patient and to introduce low-pressure suction thereto, and
a low pressure generating element operatively coupled to the treatment head controlled by a processing unit so as to introduce predefined, advantageously, pulsating pressure variation to a suction opening arranged at the treatment head.

17. The electrically powered, portable treatment apparatus of claim 16, wherein the diameter of the suction opening of the head, preferably of the cup thereat, falls within the ranges of about 10 mm through about 85 mm.

18. The electrically powered, portable treatment apparatus of claim 16, wherein the processing unit is configured to cause the apparatus to produce the pulsating pressure variation comprising low pressure pulses substantially in the range from about half a second to two seconds, preferably about one second, in duration and/or from about 5 mmHg to 350 mmHg, advantageously from about 20 mmHg to 130 mmHg, in strength.

19. The electrically powered, portable treatment apparatus of claim 16, wherein the processing unit is configured to cause the apparatus to produce the pulsating pressure variation comprising, in addition to low frequency pulses, simultaneous higher frequency oscillation, preferably falling within a range of about 5 to about 100 Hz.
